(19) United States
(12) Patent Application Publication
(10) Pub. No.: US 2026/0306570 A1
(43) Pub. Date: Oct. 8, 2026

(54) PRODUCTION OF A PHARMACEUTICAL DOSAGE FORM FROM HORSERADISH AND NASTURTIUM

(71) Applicant: KWIZDA PHARMA GMBH, Wien (AT)

(72) Inventors: Anja Witternigg, Wien (AT); Silke Feichtinger, Wien (AT); Wolfgang Reiss, Wien (AT)

(21) Appl. No.: 19/483,059

(22) PCT Filed: May 17, 2024
(86) PCT No.: PCT/EP2024/063724

§ 371 Date: Nov. 10, 2025

(30) Foreign Application Priority Data
May 17, 2023 (EP) 23174069.7

(51) Int. Cl. A61K 36/31; A61K 9/48

(52) U.S. Cl. CPC A61K 36/31; A61K 9/4825; A61K 9/4866; A61K 2236/33

(57) ABSTRACT

Disclosed is a method for producing a pharmaceutical dosage form, comprising the following steps: providing a dry extract component of horseradish (Armoracia rusticana) and nasturtium (Tropaeolum majus) with very low endogenous myrosinase activity; providing a dry drug component with horseradish root (Armoraciae rusticanae radix) and/or nasturtium herb (Tropaeoli maji herba); dry mixing the extract component with the drug component; and providing the mixture in the pharmaceutical dosage form. Also disclosed is a dosage form obtainable by this method.

### CROSS-REFERENCE TO RELATED APPLICATIONS

This application is the U.S. National Stage Application of PCT/EP2024/063724 filed May 17, 2024, claiming the benefit of priority from European application 23174069.7 filed May 17, 2023, the entire disclosure of both applications is herein incorporated by reference.

### TECHNICAL FIELD

The field of the present invention is that of the method for producing pharmaceutical dosage forms from horseradish (Armoracia rusticana) and nasturtium (Tropaeolum majus).

### BACKGROUND

Horseradish is a plant species from the genus Armoracia within the family of the cruciferous plants (Brassicaceae), which in turn is a member of the order of the cruciferous species (Brassicales). Its root (Armoraciae rusticanae radix) has long been known in herbal medicine. It is still used today—including in powder form—in herbal medicines for colds and urinary tract infections.

Also a representative of the cruciferous species is the large nasturtium (here “nasturtium” is used as a synonym) from the genus Tropaeolum within the family of the nasturtium plants (Tropaeolaceae). Nasturtium herb (Tropaeoli maji herba) has long been used in herbal medicine and is used, for example, in bladder infections.

In addition to other valuable ingredients, such as flavonoids and vitamins, both of these plants contain glucosinolates as pharmaceutically significant constituents. These glucosinolates come into contact (for example, when crushing or chewing plant material) with the enzyme myrosinase (EC 3.2.1.147, formerly EC 3.2.1.1; also called sinigrinase or thioglucosidase) located in other compartments; the enzymatic reaction forms a large number of compounds such as isothiocyanates (mustard oils), nitriles, thiocyanates, epithionitriles and oxazolidines. Isothiocyanates are of central pharmacological significance. In contrast to their starting substances, these are biologically active, for example antimicrobial and anticarcinogenic.

The antibacterial effects of isothiocyanates from horseradish are described, for example, in Negro, et al, “Phytochemical and functional analysis of horseradish (Armoracia rusticana) fermented and non-fermented root extracts.” Fitoterapia 162 (2022): 105282. The antibacterial effects of the isothiocyanates from nasturtium are dealt with, for example, in the following document: Vrca, et al. “Chemical Composition and Biological Activity of Essential Oil and Extract from the Seeds of Tropaeolum majus L. var. altum.” Food Technology and Biotechnology 60 (2022): 4.

In Conrad et al. (Arzneimittelforschung 56.12 (2006): 842-849), in vitro studies were carried out on the antibacterial effectiveness of a combination of nasturtium herb and horseradish root. A broad antibacterial effect of this combination against clinically relevant bacterial species was observed.

As a traditional herbal medicine with nasturtium herb and horseradish root, the preparation “ANGOCIN® Anti-Infekt Uro+Grippal” film-coated tablets (pharmaceutical manufacturer: Repha GmbH, Germany) is available in pharmacies. It is used for colds and upper respiratory tract infections as well as uncomplicated infections of the lower urinary tract (cystitis). However, this preparation has the disadvantage that up to 25 film-coated tablets have to be taken per day—the standard dose for an adult is already 12 film-coated tablets per day. This is detrimental to patient compliance.

### SUMMARY

It is therefore an object of the present invention to provide a production method for a preparation of nasturtium herb and horseradish root in which the valuable ingredients (in particular glucosinolates) are present in a more concentrated form. This can reduce the amount or number of dosage forms administered per day. This entails, among other things, the advantages of increasing patient compliance and reducing the effort for packaging the dosage forms (which can be considerable due to the requirements of Good Manufacturing Practice—GMP).

The present invention provides a method for producing a preferably solid pharmaceutical dosage form. This includes the following steps:
providing a dry extract component from horseradish (in particular from horseradish root) and nasturtium (in particular from nasturtium herb) with an (endogenous) myrosinase activity below 0.05 U/g, preferably below 0.01 U/g, in particular below 0.001 U/g;
providing a dry drug component with horseradish root and/or nasturtium herb;
dry mixing the extract component with the drug component; and
providing the mixture in the pharmaceutical dosage form.

It is understood that this method may include further steps.

In addition, the invention provides a preferably solid pharmaceutical dosage form which is producible by this method.

In a further aspect, the invention relates to a preferably solid pharmaceutical dosage form comprising a (dry) extract component—i.e. a component containing at least one plant extract—from horseradish (in particular from horseradish root) and nasturtium (in particular from nasturtium herb) and a drug component—i.e. a component containing at least one plant drug—with horseradish root and/or nasturtium herb.

These dosage forms may contain other components (e.g. other, non-vegetable active ingredient components). Additionally or alternatively, the extract component may contain further constituents (e.g., extracts from other plants and/or excipients) or the drug component may contain further constituents (e.g., other herbal drugs, for example, in powder form, and/or excipients).

### DETAILED DESCRIPTION

In the course of the present invention, it has surprisingly been found that when producing an organic extract from horseradish or nasturtium (in particular in an alcoholic extraction such as methanolic extraction, especially with methanol>98%), not enough myrosinase activity is retained to convert the glucosinolates present in the extract (in the gastrointestinal tract after ingestion) into the biologically active isothiocyanates. Faced with this challenge, the inventors developed the present invention based on the addition of the (raw)drug to the extract to ensure sufficient myrosinase activity (in the gastrointestinal tract after ingestion). A particular advantage of the invention is that by adding the drug (instead of, for example, adding purified myrosinase, which may not be available in a form that corresponds to the endogenous form of horseradish or nasturtium and/or does not meet the highest production standards), the rich, original active ingredient profile is better preserved and the end product is closer to the original drug combination that has been tried and tested for decades.

In the prior art, the addition of myrosinase to glucosinate-containing plant material is only described in general terms, so that the person skilled in the art cannot obtain any information on the solution according to the invention from it:

WO 2009/012485 A1 (corresponding US application: US 2010/317518A1) relates to methods for converting glucosinolate into a glucosinolate-containing plant material. That method comprises the following steps: providing an amount of processed glucosinolate-containing plant material; providing an amount of glucosinolate converting enzyme activity; mixing the processed material with the amount of enzyme activity; hydrating the mixture; and incubating the hydrated mixture, wherein the glucosinolates are enzymatically converted. Further disclosed are herbicides, fungicides, insecticides, bacteriostatic or bactericidal, cosmetic or pharmaceutical compositions based on this method. However, the document does not mention that the last two steps of that method may be carried out in vivo. It is also in no way suggested that the extracts and drugs mentioned herein may be present together in one dosage form.

U.S. Pat. No. 9,486,434 B2 (or the application US 2013/072554 A1 on which that patent is based) relates to a method for administering isothiocyanates for the therapy of bladder cancer. Disclosed is, among other things, a composition containing a mixture of baked broccoli seeds and mustard seeds. However, it is in no way suggested in the document that the extracts and drugs mentioned herein may be present together in one dosage form. Neither does it mention the nasturtium.

U.S. Pat. No. 6,824,796 B2 discloses a method for extracting nonpolar isothiocyanate from a plant comprising providing fresh or dehydrated plant material containing at least one glucosinolate forming a corresponding nonpolar isothiocyanate and endogenous myrosinase enzyme. The plant material is mixed with water and ground, and then oil is added to the water-plant material mixture. This mixture is then homogenized, whereupon the oil phase is separated from the water phase and the solids, so that the non-polar isothiocyanate is distributed in the oil phase. A composition produced by the method and disclosed as stable contains at least one nonpolar isothiocyanate and a vegetable oil. A dietary supplement may be formulated from the composition. However, it is not suggested that the extracts and drugs mentioned herein may be present together in one dosage form.

U.S. Pat. No. 5,882,646 A relates to a dietary supplement from Brassica and a method for its production. The supplement may contain broccoli and exogenous myrosinase. There is no mention of extracts, nor is there any mention of nasturtium.

US 2020/0100532 A1 discloses a powdered foodstuff comprising a mixture of a micronized powder containing active myrosinase and a glucosinolate-containing powder. However, it is in no way suggested in the document that the extracts and drugs mentioned herein may be present together in one dosage form. Horseradish and nasturtium are also not mentioned.

US 2008/0311192 A1 (corresponding Brazilian application: BR P10801686 A2) discloses a method for converting glucosinolate into isothiocyanates in the small intestine, which comprises the oral administration of an enteric-coated precursor composition of a chemoprotective agent to an individual. However, it is in no way suggested in the document that the extracts and drugs mentioned herein may be present together in one dosage form. Horseradish and nasturtium are also not mentioned.

Glucosinolates can be divided into three classes based on the structure of the different amino acid precursors: (1) aliphatic glucosinolates derived from methionine, isoleucine, leucine or valine, (2) aromatic glucosinolates derived from phenylalanine or tyrosine, and (3) indole glucosinolates derived from tryptophan (Radojčić Redovniković, et al. “Glucosinolates and their potential role in plant.” Periodicum biologorum 110.4 (2008): 297-309).

30 different glucosinolates have now been identified in horseradish (Agneta, et al. “Horseradish (Armoracia rusticana), a neglected medical and condiment species with a relevant glucosinolate profile: a review.” Genetic resources and crop evolution 60.7 (2013): 1923-1943.), where the aliphatic glucosinolate dominates sinigrin. The extract component (which is used in the method according to the invention or is contained in the dosage form according to the invention) thus contains sinigrin. The dosage form (or the extract component alone) preferably contains at least 0.1 mg, preferably at least 0.5 mg, more preferably at least 1 mg, in particular at least 2 mg of sinigrin.

In a particularly preferred embodiment, the extract component (which is used in the method according to the invention or is contained in the dosage form according to the invention) also contains at least one, preferably at least two, even more preferably at least three, in particular at least four further glucosinolates selected from the group consisting of: Glucoviorylin, glucoputranjivin, glucoarabidopsithalin, glucoiberverin, glucoiberin, glucocheirolin, glucoconringianin, gluconapin, glucocappariflexin, glucoerucin, glucokohlrabiin, glucobrassicanapin, glucomoracialapathin, glucoberteroin, glucowasabiamin, glucolesquerellin, gluconasturtiin, glucoarmoracialapicin and glucoarmoracialafolicin (cf. Agneta et al, Table 2). In particular, these are selected from the group consisting of: Glucocheirolin, gluconapin, glucobrassicanapin and gluconasturtiin.

In contrast, aromatic glucosinolates strongly dominate the nasturtium. To a large extent, glucotropaeolin (benzyl glucosinolate) makes up the glucosinolate content of the nasturtium, in addition to which sinalbin is also present (Jakubczyk, et al. “Garden nasturtium (Tropaeolum majus L.)—a source of mineral elements and bioactive compounds.” Roczniki Państwowego Zakładu Higieny 69.2 (2018)). The extract component (which is used in the method according to the invention or is contained in the dosage form according to the invention) thus also contains glucotropaeolin and preferably also sinalbin. Preferably, at least 0.05 mg, preferably at least 0.1 mg, in particular at least 0.5 mg of glucotropaeolin are present in the dosage form (or alone in the extract component).

Overall, the glucosinolate content of the dosage form according to a preferred embodiment is at least 0.15 mg, preferably at least 0.5 mg, more preferably at least 1 mg, in particular at least 2 mg. Additionally or alternatively, it is particularly preferred if the dosage form (as a single dose) forms at least 10 μg, preferably at least 20 μg, more preferably at least 40 μg, even more preferably at least 80 μg isothiocyanates in aqueous solution at pH 1 at a temperature of 37° C. within 120 minutes.

The method according to the invention may comprise further steps, for example joint compression (e.g. to form pellets) after dry mixing, whereby a compressed mixture is obtained. In other words, after dry mixing, further processing steps may be provided to ultimately obtain the mixture for the pharmaceutical dosage form.

Expediently, the dry mixing is a dry powder mixing, i.e. the extract component and the drug component are in powder form in this case and the two powders (optionally with further powders, e.g. powdered excipients) are mixed with one another.

Dry mixing has the advantage, among other things, that the premature conversion of the glucosinolates to isothiocyanates by myrosinase is avoided as far as possible. It is therefore also advisable to keep the residual moisture of the dry powders or the air humidity as low as possible during dry mixing.

The dry extract component may contain further constituents (e.g. extracts from other plants and/or excipients) or the drug component may contain further constituents (e.g. other herbal drugs, for example in powder form, and/or excipients). However, it is preferred if the extract component is obtained only from horseradish (in particular horseradish root) and nasturtium (in particular nasturtium herb).

In a preferred embodiment, providing the extract component comprises extracting (from horseradish root and nasturtium herb and optionally from parts of other plants, in particular of the order Brassicales) with an extractant. This extractant preferably comprises an organic solvent, preferably alcohol, in particular methanol. Particularly good results were achieved with methanol, since the spectrum of ingredients was thus as close as possible to the spectrum of ingredients of the crude drugs and, in addition, the yield was very high (especially with 98% methanol). Other suitable organic solvents include n-hexane, diethyl ether, tetrahydrofuran, dioxane and acetone.

The extractant preferably comprises at least 80% (v/v), preferably at least 90% (v/v), more preferably at least 95% (v/v), even more preferably at least 97.5% (v/v), in particular at least 98% (v/v) or even at least 99% (v/v) alcohol; the remaining part usually being formed by water. The alcohol is preferably methanol or ethanol.

The extraction may be carried out by different processes, such as maceration, percolation, or repercolation or other countercurrent methods, but is not limited to the methods mentioned. For example, it may be extracted for 5 hours at 25-30° C. However, this extraction time and temperature may be significantly undershot or exceeded.

In order to obtain the dry extract component, the extractant may be removed by a suitable method. These may be, for example, spray drying or drying by means of a belt dryer, but other drying methods may also be used. The drying parameters are preferably selected in such a way that the extractant is removed down to a residual amount specified in the applicable regulations (Ph.Eur., USP, etc.).

It is particularly expedient if the extract component is an extract powder. This can be obtained after extraction with the extractant by allowing the extractant to evaporate (e.g. in a rotary evaporator) so that the extract powder remains in the vessel.

In order to improve the flowability of the extract powder or, depending on the extraction method, to achieve the powder form of the extract component at all (for filling, e.g. in hard gelatin capsules), suitable excipients (e.g. maltodextrin, colloidal silicon dioxide, calcium silicate or corresponding commercially available silicate mixtures, etc.) may be added. The amount added is preferably 30% by weight, but may also be higher or lower.

According to a further preferred embodiment, the extract component is a mixed extract of horseradish (in particular horseradish root) and nasturtium (in particular nasturtium herb), and optionally of other plant constituents. Mixed extraction (i.e. the extraction from a mixture, such as a powder mixture, of the two drugs and, if necessary, other herbal drugs) entails a simplification of the production method, and it has been shown that this is possible with horseradish root and nasturtium herb without disturbing the extraction process (e.g. due to premature degradation of products). Mixed extraction may be carried out, for example, on a drug mixture with a weight ratio of horseradish root to nasturtium herb of 80 to 200. Alternatively, the extract component may also be, for example, a (powder)mixture of various (vegetable) extracts, in particular of horseradish root and nasturtium herb.

In a particularly preferred embodiment, the drug component contains horseradish root and nasturtium herb (and optionally other constituents, in particular excipients). The drug component preferably consists of horseradish root and nasturtium herb (e.g. in powder form) and, if appropriate, excipients (such as fillers or stabilizers); in other words, the drug component preferably contains no further herbal drugs (or no further active ingredients at all).

In a further preferred embodiment, the drug component contains at least enough horseradish root and/or nasturtium herb with myrosinase activity that the mixture has a myrosinase activity of at least 0.01 U/g or even at least 0.05 U/g, preferably at least 0.1 U/g, more preferably at least 0.5 U/g, even more preferably at least 1.0 U/g (based on the dry matter of the drug component).

The drug component is expediently present in powder form.

According to a particularly preferred embodiment, the extract component and the drug component are obtained from the same plant parts (namely, in particular, horseradish root and nasturtium herb). This simplifies quality control during production, as no variation between different batches (or even different plant species) has to be taken into account for the production of a batch of dosage forms. It is particularly preferred if the extract component is obtained from the same powder mixture (same batch) as the drug component (in other words: the weight ratio of horseradish root and nasturtium herb in the extraction is the same as in the drug component). For example, 1000 kg horseradish root and 1000 kg nasturtium herb (each already in powder form) are used as starting material (weight ratio: 1:1). These two powders are mixed together. Thereafter, for example, 1-10% by weight of the powder mixture is used for the extraction and, for example, a powdered mixed extract, i.e. the extract component, is produced with methanol. The remaining 90-99% by weight of the powder mixture forms the drug component. Finally, the two powders (i.e. the extract powder and the drug powder) are filled together, for example, into capsules (the dosage form).

In a further preferred embodiment, the extract component is present as an organic extract, preferably alcoholic extract, in particular methanolic or ethanolic extract.

In a preferred embodiment (in the drug component), the dosage form has a myrosinase activity of at least 0.01 U/g or even at least 0.05 U/g, preferably at least 0.1 U/g, more preferably at least 0.5 U/g, even more preferably at least 1.0 U/g (based on the dry mass of the ingredients of the dosage form, i.e. e.g. without taking into account the intrinsic mass of the capsule or sachet).

According to another preferred embodiment, the extract component and/or the drug component are in powder form. It is particularly advantageous if the extract component and the drug component are present together in a powder mixture (e.g. in a capsule). This promotes the efficient conversion of the glucosinolates by the myrosinase in the patient's gastrointestinal tract after ingestion.

For example, this powder mixture may be mixed with suitable excipients (flow regulators, anti-adhesives) in a suitable mixer to produce the dosage form, and then filled with suitable equipment in hard capsules (hard gelatin, cellulose, etc.).

In another preferred embodiment, the dosage form is a capsule, tablet, or sachet. For example, if the dosage form is a capsule, its shell may consist substantially of hard gelatin. Alternatively, by pasting the drug and extract component with a suitable oil, a pasty mixture may be produced, which can then be filled into soft gelatin capsules.

According to a further embodiment, anhydrous granulation (e.g. ethanol, methanol, etc.) may be used to produce granules from the drug/extract mixture, optionally with excipients, wherein the granules may be formulated directly (e.g. by filling into sachets) or may be compressed into tablets. These tablets may also be coated with suitable shells.

Furthermore, using solvents or dispersants (water content<4%), liquids such as solutions or suspensions could be formulated, which may be filled into bottles or other suitable dosage forms (liquid pouch, etc.).

The dosage form (in particular the extract component or the drug component) may comprise one or more (pharmaceutically acceptable) excipients. For example, a substance selected from the group of polysaccharides, such as cellulose (e.g. microcrystalline cellulose), hydroxypropylmethylcellulose, carboxymethylcellulose, hydroxyethylcellulose, ethylhydroxyethylcellulose, hydroxypropylcellulose, cellulose acetyl phthalate, hydroxypropylmethylcellulose phthalate, cellulose acetate, cellulose propionate, cellulose acetate butyrate, ethylcellulose, guar flour, alginic acid and/or alginates, pectin, polyvinylpyrrolidone, polyvinyl acetate, polyvinyl alcohol, polyvinyl chloride, polymers of acrylic acid and its salts or polyacrylates or polymethacrylates or copolymers thereof (e.g. Eudragit® E, Eudragit® R, Eudragit® S, Eudragit® NE, Eudragit® RS, Eudragit® RL), vinylpyrrolidone-vinyl acetate copolymers, nylon, colloidal silica (e.g. Aerosil®), behenates, stearates, polyamide, polyacrylamide, polyethylene, polyalkylene glycols such as polyethylene glycol, copolymers of polyalkylene glycols, e.g. polyethylene glycol and polypropylene glycol (Pluronic®, BASF), sugar, sugar alcohol and mixtures thereof. In particular, if the dosage form is a tablet, it may contain, for example, a filler such as lactose, a disintegrant such as microcrystalline cellulose, a flow agent such as colloidal silica and/or a lubricant such as magnesium stearate as excipients.

In a further preferred embodiment, the dosage form is a sustained-release dosage form (i.e. a sustained-release dosage form) such as a sustained-release tablet. How a dosage form can be formulated in sustained-release form is known in the prior art, for example from WO 2010/028794 A1 or WO 2010/105824 A1. In the embodiment according to the invention, a polymer is preferably used as a sustained-release excipient, the polymer being selected from the neutral homopolymers and copolymers of (meth)acrylic acid esters, cationic homopolymers and copolymers of (meth)acrylic acid esters with quaternary ammonium groups, polyvinyl acetate, cellulose acetate, cellulose propionate, cellulose acetate butyrate, ethyl cellulose, preferably from neutral homopolymers and copolymers of (meth)acrylic acid esters, in particular a copolymer of ethyl acrylate and methyl methacrylate.

The dosage form according to the present invention is particularly well suited for the prevention or treatment of a cold, in particular a upper respiratory tract infection, or a urinary tract infection, in particular infection-related cystitis. When administered to a patient, the dosage form preferably releases at least 1 μg, preferably at least 5 μg, more preferably at least 10 μg, in particular at least 20 or even at least 40 μg of isothiocyanates in the gastrointestinal tract of the patient in order to achieve a prophylactic or therapeutic effect.

It is evident to the person skilled in the art that the presence of a certain residual moisture content cannot be ruled out in the case of a “dry” component (such as a dry powder or dry powder) or in the case of dry mixing. The residual moisture of the extract component or of the drug component (or of the total ingredients of the dosage form according to the present invention) is preferably not more than 10% by weight and in particular not more than 5% by weight.

In order to increase the storage stability, it is expedient if the dosage form is stored in a moisture-protected manner. Therefore, in a preferred embodiment, the dosage form is packaged in a coldform blister, an aluminum-aluminum blister, a high-barrier plastic blister, or a blister containing polyvinylidene chloride.

The term “powder” (as used herein by itself or in terms such as “powder mixture”, “powder form”, or the like) is to be understood broadly and includes at least all flowable granular materials, in particular, flowable powders, powders in the narrower sense, and granules. The median particle diameter may be between 1 μm and 1000 μm, for example.

The unit (U) used herein for myrosinase activity is defined as follows: 1 U myrosinase activity is that myrosinase activity that hydrolyzes 1 pmol of sinigrin at 37° C. in one minute. This can be measured either by means of sinigrin degradation (photometrically determinable at 230 nm) or glucose release (determinable by coupled enzyme assay) (cf. Dosz, et al. “Total myrosinase activity estimates in brassica vegetable produce.” Journal of agricultural and food chemistry 62.32 (2014): 8094-8100.). The determination of myrosinase activity via sinigrin degradation is preferred, in particular as carried out in Example 3.

Furthermore, the present invention relates to the following embodiments:

Embodiment 1. A method for producing a pharmaceutical dosage form, comprising the steps of:
providing a dry extract component of horseradish (Armoracia rusticana) and nasturtium (Tropaeolum majus) with an endogenous myrosinase (EC 3.2.1.147) activity below 0.05 U/g, preferably below 0.01 U/g, in particular below 0.001 U/g;
providing a dry drug component with horseradish root (Armoraciae rusticanae radix) and/or nasturtium herb (Tropaeoli maji herba);
dry mixing the extract component with the drug component; and
providing the mixture in the pharmaceutical dosage form.

Embodiment 2. The method according to embodiment 1, wherein the drug component contains at least enough horseradish root and/or nasturtium herb with myrosinase activity that the mixture has a myrosinase activity of at least 0.01 U/g or even at least 0.05 U/g, preferably at least 0.1 U/g, more preferably at least 0.5 U/g, even more preferably at least 1.0 U/g.

Embodiment 3. The method according to embodiment 1 or 2, wherein the drug component includes horseradish root (Armoraciae rusticanae radix) and nasturtium herb (Tropaeoli maji herba).

Embodiment 4. The method according to any one of embodiments 1 to 3, wherein the extract component is a mixed extract of horseradish (Armoracia rusticana) and nasturtium (Tropaeolum majus), and optionally other plant constituents.

Embodiment 5. The method according to any one of embodiments 1 to 4, wherein the extract component is an extract powder.

Embodiment 6. The method according to any one of embodiments 1 to 5, wherein providing the extract component comprises extracting with an extractant comprising an organic solvent, preferably alcohol, in particular methanol.

Embodiment 7. The method according to embodiment 6, wherein the extractant comprises at least 80% (v/v), preferably at least 90% (v/v), more preferably at least 95% (v/v), even more preferably at least 97.5% (v/v), in particular at least 98% (v/v) or even at least 99% (v/v) alcohol, preferably wherein the alcohol is methanol or ethanol.

Embodiment 8. The method according to any one of embodiments 1 to 7, wherein the dry mixing is dry powder mixing.

Embodiment 9. The method according to any one of embodiments 1 to 8, wherein the drug component is in powder form.

Embodiment 10. The method according to any of embodiments 1 to 9, wherein the extract component and the drug component are obtained from the same plant parts.

Embodiment 11. A pharmaceutical dosage form, producible by the method according to one of embodiments 1 to 11.

Embodiment 12. A pharmaceutical dosage form comprising:
an extract component of horseradish (Armoracia rusticana) and nasturtium (Tropaeolum majus); and
a drug component with horseradish root (Armoraciae rusticanae radix) and/or nasturtium herb (Tropaeoli maji herba).

Embodiment 13. The pharmaceutical dosage form according to one of embodiments 11 and 12, wherein the dosage form has a myrosinase activity of at least 0.01 U/g or even at least 0.05 U/g, preferably at least 0.1 U/g, more preferably at least 0.5 U/g, even more preferably at least 1.0 U/g.

Embodiment 14. The pharmaceutical dosage form according to any of embodiments 11 to 13, wherein the extract component and/or the drug component are in powder form.

Embodiment 15. The pharmaceutical dosage form according to any of embodiments 11 to 14, wherein the extract component and the drug component are present together in a powder mixture.

Embodiment 16. The pharmaceutical dosage form according to any of embodiments 11 to 15, wherein the dosage form is a solid dosage form such as a capsule, tablet, or sachet.

Embodiment 17. The pharmaceutical dosage form according to any of embodiments 11 to 16, wherein the dosage form contains at least 0.15 mg of glucosinolates.

Embodiment 18. The pharmaceutical dosage form according to any of embodiments 11 to 17, wherein the dosage form forms at least 10 μg, preferably at least 20 μg, more preferably at least 40 μg, even more preferably at least 80 μg isothiocyanates in aqueous solution at pH 1 at 37° C. within 120 minutes.

Embodiment 19. The pharmaceutical dosage form according to any of embodiments 11 to 18, wherein the extract component is a mixed extract of horseradish (Armoracia rusticana) and nasturtium (Tropaeolum majus), and optionally of other plant constituents.

Embodiment 20. The pharmaceutical dosage form according to any of embodiments 11 to 19, wherein the extract component is an extract powder.

Embodiment 21. The pharmaceutical dosage form according to any of embodiments 11 to 20, wherein the extract component is an organic extract, preferably an alcoholic extract, in particular a methanolic or ethanolic extract.

Embodiment 22. The pharmaceutical dosage form according to any of embodiments 11 to 21, wherein the drug component includes horseradish root (Armoraciae rusticanae radix) and nasturtium herb (Tropaeoli maji herba).

Embodiment 23. The pharmaceutical dosage form according to any of embodiments 11 to 22, for use in the prevention or treatment of a cold, in particular a upper respiratory tract infection, or a urinary tract infection, in particular infection-related cystitis, of a patient.

Embodiment 24. The pharmaceutical dosage form for use according to embodiment 23, wherein the dosage form releases at least 1 μg, preferably at least 5 μg, more preferably at least 10 μg, in particular at least 20 or even at least 40 μg of isothiocyanates in the gastrointestinal tract of the patient.

The invention is explained in more detail below with reference to preferred, non-limiting examples.

### Example 1—Extraction Tests

80 parts Armoraciae rusticanae radix and 200 parts Tropaeoli maji herba were comminuted to a particle size suitable for extraction (mass ratio 80:200). The two drugs prepared in this way were then homogeneously mixed in a mixer. The myrosinase activity in this drug mixture, measured according to Example 3, was 1.0 U/g. The drug mixture contained about 6000 mg/kg sinigrin and about 1800 mg/kg glucotropaeolin.

The extraction was tested with various organic solvents (extraction: 5 h at 25-30° C.), the results are shown in Table 1.

TABLE 1

| | Average yield solids | Resulting drug-extract |
|---|---|---|
| Extractants | [mg] | ratio |
| n-Hexane | 104 | 83.3:1 |
| Diethyl ether | 190 | 45.6:1 |
| Tetrahydrofuran | 218 | 39.8:1 |
| Dioxane | 259 | 33.5:1 |
| Acetone | 255 | 33.9:1 |
| Ethanol 96% | 410 | 21.2:1 |
| Methanol 98% | 1201 | 7.2:1 |

Consequently, methanol (98%) turned out to be the most suitable extractant for this drug mixture, as the yield was the highest (and thus also the widest spectrum of different active ingredients was present).

### Example 2—Production of the Pharmaceutical Dosage Form

### Producing the Drug Component

80 parts Armoraciae rusticanae radix and 200 parts Tropaeoli maji herba are comminuted to a particle size suitable for extraction (mass ratio 80:200). The two drugs prepared in this way are then homogeneously mixed in a mixer. The myrosinase activity in this drug mixture, measured according to Example 3, is 1.0 U/g. The drug mixture contains about 6000 mg/kg sinigrin and about 1800 mg/kg glucotropaeolin. 5% by weight of this drug mixture is used as the drug component of the dosage form, the remaining part is subjected to an extraction (see next paragraph).

### Production of the Extract Component

Methanol (>98%) is placed in a suitable preparation vessel. The drug mixture is added. The batch ratio is 1:7 (w/w) (drug mixture to extractant), but may also be from 1:3 to 1:50 or greater, for example. It is extracted for 5 h at 25-30° C. After extraction, the extractant is removed by means of spray drying in order to obtain the dry, pulverulent extract component with the addition of 30% by weight maltodextrin. No myrosinase activity (measured according to example 3) is detectable in this extract. The extract contains about 35000 mg/kg sinigrin and about 10000 mg/kg glucotropaeolin (without taking into account the maltodextrin).

### Dry Mixing

The drug component and the extract component are homogeneously mixed in a mixer to form a powder mixture.

Filling into Dosage Form

The powder mixture is filled into hard gelatin capsules. The powder mixture in a capsule contains 60 mg of drug powder and 150 mg of extract powder (without taking into account the maltodextrin). One capsule (equivalent to a single dose) contains approximately 5 mg of sinigrin and 2 mg of glucotropaeolin.

### Example 3—Measurement of Myrosinase Activity

The myrosinase activity of the drug mixture is determined photometrically by the degradation of the glucosinolate sinigrin. This degradation can be determined by the decrease in absorption at 230 nm (path length cuvette 1 cm).

### Required Solutions:

Sodium chloride solution: 468 mg NaCl are dissolved in 100 mL HQ water=80 mM (neutral pH)
Substrate solution: 10 mg of sinigrin hydrate are dissolved in 2 mL H₂O.
Drug solution: 300 mg of drug (or extract) are dissolved in 5 mL of water (ice cold), centrifuged at 4300 rpm for 2 minutes (cooling the centrifuge to 4° C.). An aliquot is then filtered through a syringe filter (0.45 μm).

2.97 mL of the NaCl solution are placed in a cuvette, 15 μL of substrate solution are pipetted into it and mixed by pipetting up and down.

The cuvette is then closed and heated to 37° C. in a water bath. In the meantime, the drug solution is being produced. 15 μL of the drug solution are pipetted into the temperature-controlled cuvette and the absorption at 230 nm is measured immediately. A cuvette with pure NaCl solution is used as the blank. After measurement (T0), the cuvette is immediately returned to the water bath and measured every 5 min for a period of 30 min.

The enzyme activity is determined by the glucosinolate decrease over 30 min:

$$Myrosinase activity = \frac{1000 \times {V}_{A}}{( {V}_{sample} \times C )} \times \frac{2 \times ( {A}_{0} - {A}_{t} )}{E \times t}$$

V_{A} . . . volume of the reaction mixture in mL (3)
A₀ . . . absorption at start of measurement
Aₜ . . . absorption after time t
t . . . reaction time in min (30)
Vₛₐₘₚₗₑ . . . volume of the examined sample in mL (0.015)
C . . . concentration of the sample in g/mL (0.300/5)
E . . . molar extinction coefficient for sinigrin (7500 M⁻¹ cm⁻¹)

The myrosinase activity in unit indicates pmol glucosinolates (sinigrin) which are hydrolyzed per minute. Calculated on 1 g of drug (or extract), this results in an enzyme activity of U/g of drug (or extract).

### Example 4—Release Behaviour of the Dosage Form

The powder mixture produced according to example 1 corresponding to a single dose (i.e. the content of a capsule) was investigated with regard to the isothiocyanate release behavior.

To simulate the conditions in the gastrointestinal tract, a generally accepted method based on Pharmacopoea Europaea was used. The powder mixture was dispersed in 100 mL of water at 37° C./pH 1 (hydrochloric acid) and stirred. After 0 (control), 2 and 3 hours, samples were taken and tested for the content of allyl isothiocyanate. After 2 hours, the pH was adjusted to 7 with sodium hydroxide, and the mixture was stirred further.

After 2 hours, approx. 120 μg of allyl isothiocyanate were released from this single dose extrapolated, this value had not changed significantly at the measuring point for 3 hours.

1. A method for producing a pharmaceutical dosage form, comprising the following steps:
providing a dry extract component of horseradish (Armoracia rusticana) and nasturtium (Tropaeolum majus) with an endogenous myrosinase (EC 3.2.1.147) activity below 0.05 U/g, preferably below 0.01 U/g, in particular below 0.001 U/g;
providing a dry drug component with horseradish root (Armoraciae rusticanae radix) and/or nasturtium herb (Tropaeoli maji herba);
dry mixing the extract component with the drug component; and
providing the mixture in the pharmaceutical dosage form.

2. The method according to claim 1, wherein the drug component contains at least enough horseradish root and/or nasturtium herb with myrosinase activity that the mixture has a myrosinase activity of at least 0.01 U/g or even at least 0.05 U/g, preferably at least 0.1 U/g, more preferably at least 0.5 U/g, even more preferably at least 1.0 U/g.

3. The method according to claim 1, wherein the drug component includes horseradish root (Armoraciae rusticanae radix) and nasturtium herb (Tropaeoli maji herba).

4. The method according to claim 1, wherein the extract component is a mixed extract of horseradish (Armoracia rusticana) and nasturtium (Tropaeolum majus), and optionally other plant constituents.

5. The method according to claim 1, wherein providing the extract component comprises extracting with an extractant comprising an organic solvent, preferably alcohol, in particular methanol.

6. The method according to claim 5, wherein the extractant is at least 80% (v/v), preferably at least 90% (v/v), more preferably at least 95% (v/v), even more preferably at least 97.5% (v/v), in particular at least 98% (v/v) or even at least 99% (v/v) alcohol, preferably wherein the alcohol is methanol or ethanol.

7. The method according to claim 1, wherein the extract component and the drug component are obtained from the same plant parts.

8. A pharmaceutical dosage form producible by the method according to claim 1.

9. A pharmaceutical dosage form comprising:
an extract component of horseradish (Armoracia rusticana) and nasturtium (Tropaeolum majus); and
a drug component with horseradish root (Armoraciae rusticanae radix) and/or nasturtium herb (Tropaeoli maji herba);
Wherein the dosage form contains at least 1 mg glucosinolates.

10. The pharmaceutical dosage form according to claim 8, wherein the dosage form has a myrosinase activity of at least 0.01 U/g or even at least 0.05 U/g, preferably at least 0.1 U/g, more preferably at least 0.5 U/g, even more preferably at least 1.0 U/g.

11. The pharmaceutical dosage form according to claim 8, wherein the extract component and the drug component are present together in a powder mixture.

12. The pharmaceutical dosage form according to claim 8, wherein the dosage form is a solid dosage form such as a capsule, tablet, or sachet.

13. The pharmaceutical dosage form according to claim 8, wherein the dosage form contains at least 2 mg of glucosinolates.

14. The pharmaceutical dosage form according to claim 8, wherein the dosage form forms at least 10 μg, preferably at least 20 μg, more preferably at least 40 μg, even more preferably at least 80 μg isothiocyanates in aqueous solution at pH 1 at 37° C. within 120 minutes.

15. The pharmaceutical dosage form according to claim 8, wherein the extract component is an organic extract, preferably an alcoholic extract, in particular a methanolic or ethanolic extract.
